(19) United States
(12) Patent Application Publication
(10) Pub. No.: US 2010/0310502 A1
(43) Pub. Date: Dec. 9, 2010

(54) Method of treating local infections in mammals

(76) Inventors: Anders Linden, Molndal (SE); Elin Silverpil, Goteborg (SE); Pernilla Glader, Goteborg (SE)

Correspondence Address: LYNN E BARBER, P O BOX 16528, FORT WORTH, TX, 76162, US

(21) Appl. No.: 12/455,929

(22) Filed: Jun. 9, 2009

(51) Int. Cl. A61K 38/20; A61P 31/00

(52) U.S. Cl. 424/85.2

(57) ABSTRACT

An effective method is provided for local treatment against localized microbial infection, including abscesses, in skin and soft tissue and elsewhere in mammalian organs. The method employs mammalian IL-17 receptor agonists, which exert several antimicrobial actions, including the stimulation of neutrophil turnover as well as macrophage phagocytosis locally in an infected mammalian organ.

### BACKGROUND OF THE INVENTION

1. Field of the Invention

The present invention provides an effective method for local treatment against localized microbial infection, including abscesses, in skin and soft tissue and elsewhere in mammalian organs. The method employs the mammalian cytokines interleukin-17 (abbreviated IL-17 or, as herein this patent application, IL-17A), IL-17F and IL-17A/F, as well as synthetic agonists at IL-17 receptors including the IL-17 A/C receptor complex and other IL-17 receptors. These IL-17 receptor agonists exert several antimicrobial actions, including the stimulation of neutrophil turnover as well as macrophage phagocytosis locally in an infected mammalian organ.

2. Background of the Related Art

Interleukin-17. Interleukin (IL)-17, also named IL-17A, is a 35 kDa homodimeric molecule released from certain T lymphocytes and possibly from other inflammatory cells as well under certain conditions (1-4). IL-17A represents the archetype member of the IL-17 family of cytokines and it acts through specific receptors, including the IL-17 A/C receptor complex and, possibly, through other IL-17 receptors as well (1-6). IL-17A is currently considered to be a pro-inflammatory molecule, which is one that recruits neutrophils (neutrophilic granulocytes), accumulates these cells and their products as part of host defense in several mammalian organs (1-5). These products include antibacterial compounds such as neutrophil elastase, myeloperoxidase (MPO), and matrix metalloproteinase-9 (MMP-9) (1-4). The production and release of IL-17A is believed to be critical in mammalian host defense against several bacterial and fungal species, because of its functional position at the interface of innate and acquired immunity (1-4). This position enables it to exert an orchestrating role of a key antibacterial effector cell, the neutrophil, locally in the infected organ as well as stimulating production of antibacterial peptides such as beta-defensins (including human beta-defensin-2) and human cathelicidin (also named LL-37) in structural cells (1-4,7,8).

Based upon the existing scientific literature, it has been assumed that IL-17A acts in a pro-inflammatory and potentially detrimental manner in mammalian organs (9,10). In support of this, there are several chronic inflammatory conditions that display signs of an increase in local IL-17 protein and/or messenger RNA, including disorders in the lungs such as asthma, bronchial hyperresponsiveness and exacerbations of cystic fibrosis; gut disorders such as morbus Crohn and peptic ulcers; skin disorders such atopic dermatitis and neurologic disease such as multiple sclerosis (1,2,9,10).

Because it has previously been assumed that IL-17A, by accumulating neutrophils locally, will cause detrimental actions on organ tissue, the general notion among those familiar with the IL-17A literature has been that the action of endogenous IL-17A should be blocked to treat disease signified by excessive local neutrophil accumulation (1,2-4,9,10).

Localized infections in mammalian organs. Abscesses constitute a common type of localized bacterial infection in single organs, including skin and soft tissue, of immunocompetent or immunodeficient humans and other mammals (11,12). These abscesses are currently treated with surgical procedures. The surgical procedures include incisions and mechanically assured drainage of pus. This treatment strategy requires surgical skills of the physician and substantial healing time; two factors that contribute to cost for the health care and insurance systems. Normally, chemical agents such as antibiotics do not constitute clinically efficient therapeutic options in these types of infections, in part because of the poor vascularization in the affected area. This poor vascularization constitutes a major practical obstacle, because it leads to weak tissue penetration of the antibiotic, a fact which makes it very difficult to achieve local concentrations that are sufficiently high to exert a true bactericidal or bacteriostatic effect. In addition, the local application of antibiotics is believed to increase the risk of detrimental side effects, including allergic sensitization and clinically manifest allergy, as well as increasing the risk of bacterial resistance against the antibiotic. Sometimes, current therapeutic approaches even include surgical removal of the tissue infected by bacteria, such as in severe muscular or subcutaneous infections in the extremities or other types of soft tissue infections, as well as in bone infections or in appendicitis.

Disseminated fungal infection in single organs, including skin and soft tissue, of immunocompetent or immunodeficient humans and other mammals also constitute an example of localized infection that currently may be hard to treat using existing therapeutic approaches. For example, these disseminated infections may occur in the gut, muscle or in the lungs of mammals but also in other locations, as described below. Current therapeutic approaches frequently employ single or multiple chemical agents (i.e. fungicidal antibiotics). The obstacles with this type of fungicidal treatment are the same as described above for treatment of bacterial infections in order to gain sufficiently high local concentrations and the risk of side effects after application of high local concentrations of fungicidal antibiotics.

Based upon our own unpublished studies, from which data are included as a fundament of this patent application, we can now surprisingly conclude that IL-17A is not merely a cytokine that accumulates neutrophils and neutrophil-related activity (1-4), which would be potentially detrimental in a number of inflammatory conditions in mammals, but that IL-17A is also a cytokine that contributes to the resolution of neutrophilic inflammation. The critical evidence for this contribution to the resolution of neutrophilic inflammation is our enclosed data sets; data sets showing that stimulation of neutrophils with IL-17A induces the controlled death (apoptosis) of these critical effector cells in host defense, while at the same time maintaining the ability of the neutrophil to release an archetype antibacterial product, myeloperoxidase (MPO). Moreover, these data sets show that stimulation of macrophages with IL-17A increases macrophage phagocytosis of aged, presumably apoptotic, neutrophils. The same stimulation of macrophages with IL-17A also increases macrophage phagocytosis of latex beads. In the invention herein, we claim that IL-17 receptor agonists can be utilized as pharmacological tools for local antimicrobial treatment against localized microbial infection, including abscesses, in skin and soft tissue and elsewhere in mammalian organs. The antimicrobial effect of these IL-17 receptor agonists is achieved by increasing the turn-over of neutrophils (including their recruitment, accumulation, apoptosis and their phagocytosis by macrophages) as well as by increasing the phagocytosis of bacteria, fungi, cellular debris and particles by macrophages and the release of antimicrobial peptides locally. Finally, in the invention herein we claim that the IL-17 receptor agonists stimulate the phagocytosis of bacteria and fungi by neutrophils.

A few earlier patent applications describe the use of IL-17 for enhancing the infiltration of inflammatory cells into a tissue, for example US20070160576 A1 and WO2000020593 A1; however unlike the invention herein these applications do not describe or mention the specific use of IL-17 receptor agonists as therapy against local infections in mammals. No earlier documents describe how IL-17 receptor agonists can be used to stimulate the phagocytosis of apoptotic neutrophils, fungi, bacteria or cellular debris by macrophages.

In conclusion, none of the above prior work, taken either singularly or in combination, teaches use of IL-17 receptor agonists as described and claimed herein.

### SUMMARY OF THE INVENTION

An effective method is provided for local treatment against localized microbial infection, including abscesses, in skin and soft tissue and elsewhere in mammalian organs. The method employs mammalian IL-17 receptor agonists; receptor agonists that exert several antimicrobial actions, and the stimulation of neutrophil turnover as well as macrophage phagocytosis locally in an infected mammalian organ.

A primary object of the present invention is to utilize IL-17 receptor agonists as pharmacological tools for local treatment against localized microbial infection, including abscesses, in skin and soft tissue and elsewhere in mammalian organs by increasing the turn-over of neutrophils.

An object of the present invention is to provide IL-17 receptor agonists to induce controlled death (apoptosis) of neutrophils.

An object of the present invention is to provide IL-17 receptor agonists to induce the release of antimicrobial compounds from neutrophils.

Another object is to provide IL-17 receptor agonists to increase macrophage phagocytosis of aged, apoptotic, neutrophils.

Another object is to provide IL-17 receptor agonists to increase phagocytosis of bacteria, fungi, cellular debris and particles by macrophages.

An object of the present invention is to provide IL-17 receptor agonists to stimulate the phagocytosis of bacteria and fungi by neutrophils.

Other objects and advantages of the present invention will become obvious to the reader and it is intended that these objects and advantages are within the scope of the present invention.

### BRIEF DESCRIPTION OF THE DRAWINGS

FIG. 1 is a graph showing the expected decrease in expected size of subcutaneous abscesses in humans after intermittent local administration of recombinant human IL-17A protein at the site of the abscess.

FIG. 2 is a graph showing the impact of recombinant mouse IL-17A protein on mouse neutrophil apoptosis; where the percentage of apoptotic and viable neutrophils (% neutrophils) respectively, is presented at different concentrations of IL-17A (ng/mL).

FIG. 3 is a graph showing the impact of recombinant mouse IL-17A protein on the release of myeloperoxidase (MPO) in mouse neutrophils; where the MPO concentration (ng/mL) is presented at different concentrations of IL-17A (ng/mL).

FIG. 4 shows photographs of mouse macrophages phagocytising fluorescent mouse neutrophils (A) and (B) latex beads, after stimulation by recombinant mouse IL-17A protein (100 ng/mL).

FIG. 5 is a graph showing the effect of mouse recombinat IL-17A protein on the phagoctosis of aged mouse neutrophils by mouse macrophages; where the percent of macrophages containing fluorescence-labeled neutrophils (% phagocytosis) is presented at different concentrations of IL-17A (ng/mL).

FIG. 6 is a graph showing the effect of mouse recombinat IL-17A protein on the phagoctosis of latex beads by mouse macrophages; where the phagocytic index is presented at different concentrations of IL-17A (ng/mL).

FIG. 7 is a graph showing the effect of human recombinat IL-17A protein on the phagoctosis of latex beads by human monocyte-derived macrophages; where the phagocytic index is presented at different concentrations of IL-17A (ng/mL).

FIG. 8 is a graph showing the effect of mouse recombinat IL-17A protein on the release of macrophage inflammatory protein-2 (MIP-2) by mouse macrophages; where the concentration of MIP-2 (pg/mL) is presented at different concentrations of IL-17A (ng/mL).

### DETAILED DESCRIPTION OF THE INVENTION AND PREFERRED EMBODIMENTS THEREOF

In this patent application, we define IL-17 receptor agonists as the mammalian cytokines interleukin-17 (abbreviated IL-17 or IL-17A) IL-17F, IL-17A/F, and other synthetic or naturally-occurring agonists at IL-17 receptors including the IL-17 A/C receptor complex and other IL-17 receptors. As disclosed in detail herein, the local administration of a sterile solution of IL-17 receptor agonists at the site of the infection in a mammalian organ results in strong antimicrobial effects locally, mediated by neutrophils and macrophages mainly.

It is known from experiments in vitro that a pyrogen-free (sterile) solution with a concentration of 1 to 1000 ng/mL of an IL-17 receptor agonist will initiate the herein described antimicrobial effects. It is also known that a total dose of such an IL-17 receptor agonist as low as 1 to 3 micrograms will accumulate neutrophils locally in vivo (13-15). Based upon this knowledge, we claim that using a solution within the referred concentration range, resulting in a total dose ranging from 0.1 to 100 micrograms, will prove effective for the use of the current invention in mammals in vivo.

Accordingly, in some embodiments of the present invention, the total dose of an IL-17 receptor agonist achieving desired effects is in the range from about 0.1 microgram to about 100 microgram in vivo. Thus, the total dose can be from 0.1 microgram to 0.3 microgram, from 0.1 microgram to 1 microgram, from 0.1 microgram to 3 microgram, from 0.1 microgram to 10 microgram, from 0.1 microgram to 30 microgram, from 0.1 microgram to 100 microgram, from 0.3 microgram to 3 microgram, from 0.3 microgram to 10 microgram, from 0.3 microgram to 30 microgram, from 0.3 microgram to 100 microgram, from 1 microgram to 3 microgram, from 1 microgram to 10 microgram, from 1 microgram to 30 microgram, from 1 microgram to 100 microgram, from 3 microgram to 10 microgram, from 3 microgram to 30 microgram, from 3 microgram to 100 microgram, from 10 microgram to 30 microgram, from 10 microgram to 100 microgram, from 30 microgram to 100 microgram, and the like.

The administration of IL-17 receptor agonists can be conducted once, intermittently, repeatedly or continuously over time. This administration can be conducted subcutaneously, intramuscularly or locally in other ways well-known for those skilled in the art.

More specifically, this administration of IL-17 receptor agonists results in the following events:

### Immediate Effects:

1) Structural cells produce several cytokines that recruit neutrophils to the locus of infection, including IL-8 in humans.
2) Structural cells produce IL-6 that contributes to neutrophil activation at the locus of infection.
3) Structural cells produce granulocyte colony-stimulating factor (G-CSF) and granulocyte macrophage colony-stimulating factor (GM-CSF) that prolong the time period that neutrophils are able to fight bacteria.
4) Structural cells produce beta-defensins that exert an antibacterial action, including beta-defensin-2 and LL-37 in humans.
5) Neutrophils release antibacterial compounds including neutrophil elastase, MPO and MMP-9 into the surrounding tissue.
6) Neutrophils phagocytize bacteria and fungi.

### Long-Term Effects:

1) Macrophage precursors are recruited and differentiate into mature macrophages.
2) Neutrophils undergo controlled cell death (apotosis) and thereby do not release their tissue-toxic antibacterial armature locally.
3) Macrophages start to phagocytize aged and apoptotic neutrophils and thereby reduce pus.
4) Macrophages do not produce excessive amounts of neutrophil-recruiting cytokines and thereby do not contribute to excessive accumulation of neutrophils in the long term.
5) Macrophages phagocytize bacteria and fungi.
6) Macrophages phagocytize potentially harmful cellular debris and particles.

The net functional outcome of this local administration of IL-17 receptor agonists is that bacteria and/or fungi are killed and removed from the infectious locus, without any severe neutrophil-related tissue damage. When present, microbial abscesses shrink and resolve after local administration of IL-17 receptor agonists.

Examples of common local infections in skin, soft tissue or elsewhere in mammals that can be treated successfully with local administration of IL-17 receptor agonists through tissue injections are: abdominal abscess, appendicitis, brain abscess, breast abscess, buttock abscess, carbuncle, cellulitis, diverticulitis, empyema, encephalitis, fascitis, finger abscess, fistula, folliculitis, furuncles, generalised subcutaneous infection of the extremities, head abscess, hidradenitis, impetigo, limb abscess, mastitis, neck abscess, paronychia, pleuritis, sinusitis, spondylitis, toe abscess, trunk abscess and recurrent infections by multiresistent Staphylococcus aureus and others as obvious to a person skilled in the art.

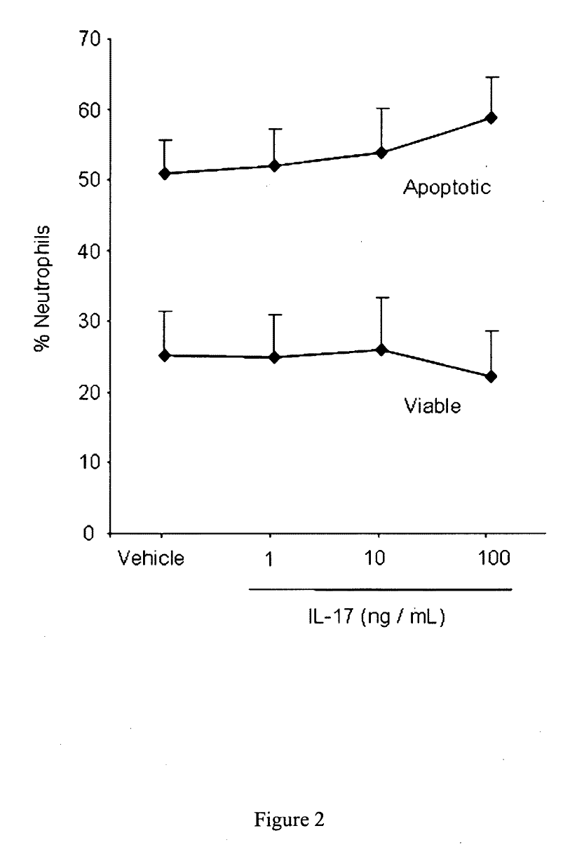

In order to show that stimulation with IL-17 receptor agonists stimulate neutrophils to undergo controlled cell death and thereby cause no severe tissue damage, an apotosis in vitro test was performed, as described in Example 3. The results are illustrated as a graph showing the impact of recombinant mouse IL-17A protein on the apoptosis (Annexin V+7AAD-cells) and viability (Annexin V−7AAD-cells) of mouse blood neutrophils after 48 hours of stimulation in vitro with different concentrations of recombinant mouse IL-17A protein. Data is presented as mean with SEM (n=6; p=0.03 for IL-17 0 versus 100 ng/mL) in FIG. 2.

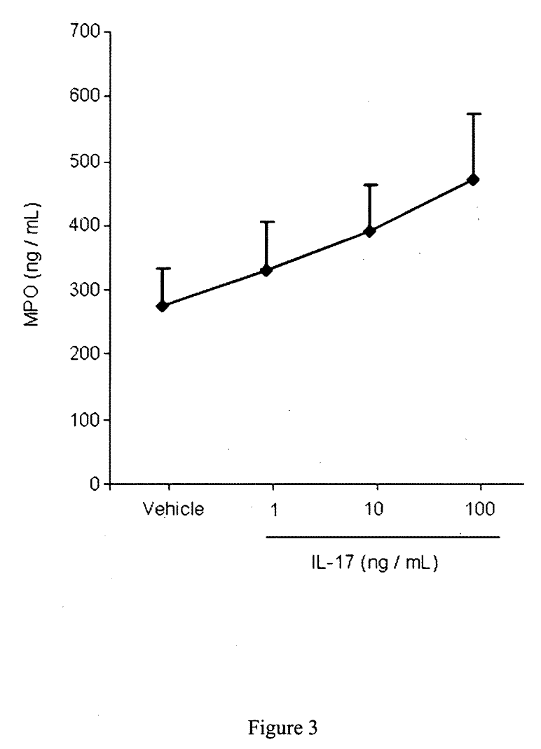

In order to show that neutrophils that undergo apoptosis during stimulation with IL-17 receptor agonists are still capable of releasing myeloperoxidase (MPO), an archetype bactericidal compound, an in vitro stimulation test was performed, as described in Example 4. The results are illustrated as a graph showing the impact of recombinant mouse IL-17A protein on the concentration of MPO in conditioned medium from mouse blood neutrophils after 48 hours of culture and stimulation in vitro. Data is presented as mean with SEM (n=4; p=0.01 for 0 ng/mL vs. maximum IL-17A-induced change in the concentration range up to 100 ng/mL) in FIG. 3.

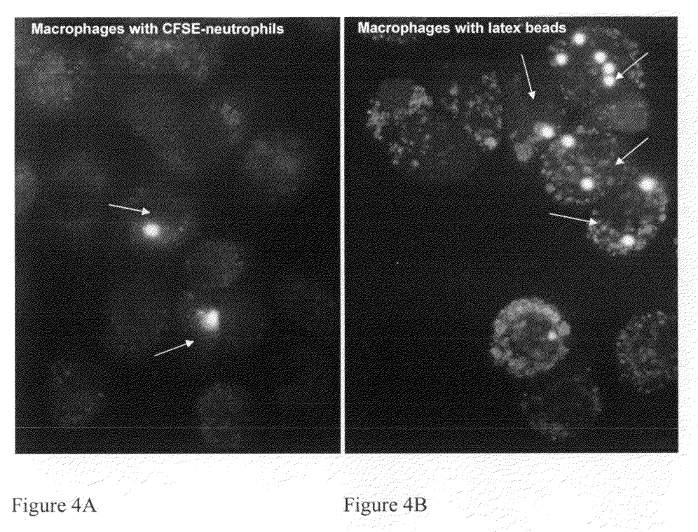

In order to show that IL-17 receptor agonists are capable of stimulating macrophages to phagocytose neutrophils and latex beads, respectively, a phagocytosis test in vitro was performed, see Examples 5 and 6. The impact of different concentrations of recombinant mouse IL-17A protein on the phagocytosis of (A) mouse blood neutrophils and (B) latex beads by mouse bronchoalveolar macrophages is shown in FIG. 4. Fluorescent (A) CFSE-labelled neutrophils (green), and (B) latex beads (green), are indicated by arrows. The nuclei of the macrophages are labelled red with 7AAD (magnification ×100).

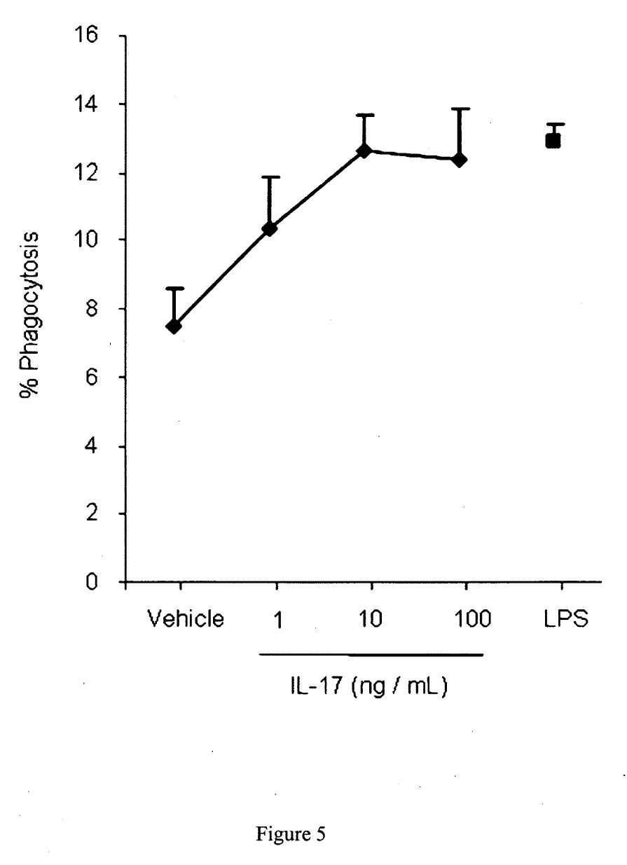

In order to show that IL-17 receptor agonists are capable of stimulating macrophages to phagocytize neutrophils, a quantification of the repeated phagocytosis test in vitro was performed as well, see Example 5. The results are illustrated in FIG. 5 as a graph showing phagocytosis of aged mouse blood neutrophils by mouse bronchoalveolar macrophages after 24 hours of pre-stimulation in vitro with different concentrations of recombinant mouse IL-17A protein or the positive control, endotoxin (LPS from E. coli). Data is presented as mean with SEM (n=4; p=0.01 for 0 ng/mL vs. maximum IL-17A-induced change in the concentration range up to 100 ng/mL).

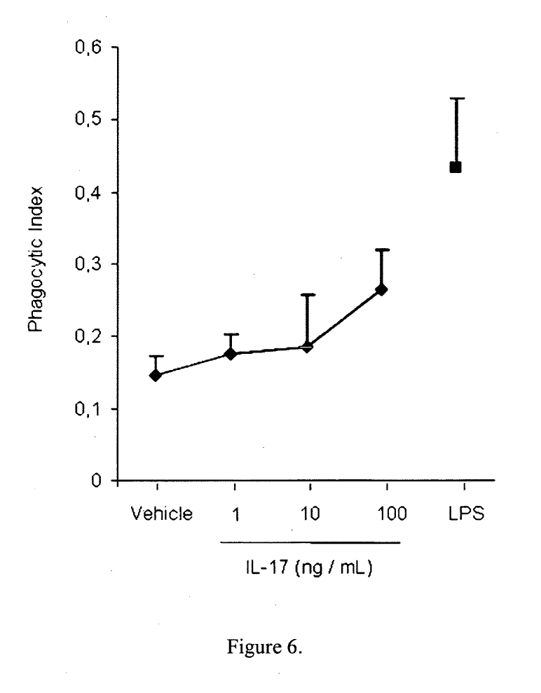

In order to show that IL-17 receptor agonists are capable of stimulating phagocytosis of particles by macrophages, a quantification of the repeated phagocytosis test in vitro was performed, see Example 6. The results are illustrated in FIG. 6 as a graph showing the phagocytosis of fluorescence-labeled latex beads by mouse bronchoalveolar macrophages after 24 hours of pre-stimulatin in vitro with different concentrations of recombinant mouse IL-17A protein or the positive control, endotoxin (LPS from E. coli). Data is presented as mean with SEM (n=5; p=0.005 for 0 ng/mL vs. maximum IL-17A-induced change in the concentration range up to 100 ng/mL).

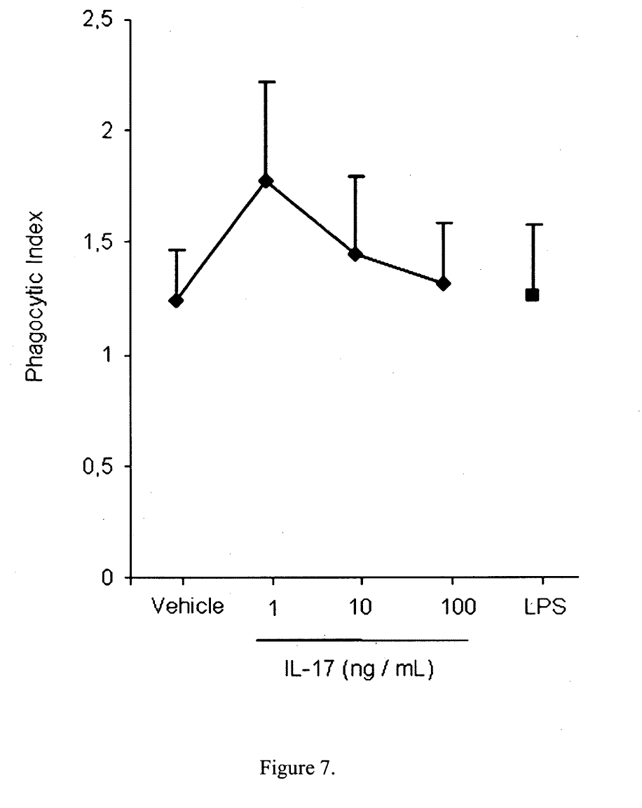

In order to show that IL-17 receptor agonists are capable of stimulating macrophage phagocytosis in humans, a quantitative in vitro test was performed, see Example 7. The results are illustrated in FIG. 7 as a graph showing the phagocytosis of fluorescence-labeled latex beads by human monocyte-derived macrophages after 24 hours of pre-stimulation in vitro with different concentrations (ng/mL) of recombinant human IL-17A protein or the positive control, endotoxin (LPS from E. coli). Data is presented as mean with SEM (n=4; p=0.01 for 0 ng/mL vs. maximum IL-17A-induced change in the concentration range up to 100 ng/mL).

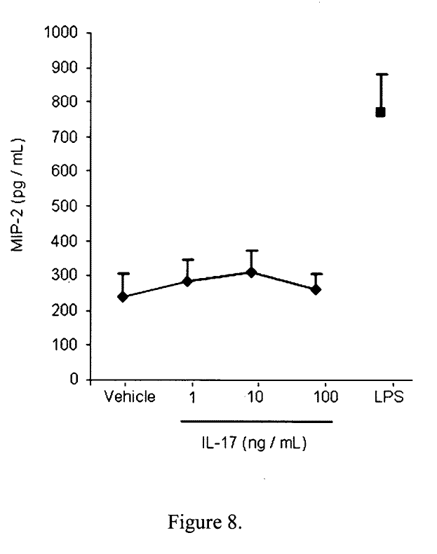

In order to show that stimulation of macrophages with IL-17 receptor agonists does not lead to an excess release of neutrophil-recruiting cytokines, an in vitro test was performed, see Example 8. The results are illustrated in FIG. 8 as a graph showing concentrations of macrophage inflammatory protein (MIP)-2 protein in conditioned medium from bronchoalveolar mouse macrophages after 24 hours of culture in vitro, during stimulation with different concentrations (ng/mL) of recombinant mouse IL-17A protein or the positive control, endotoxin (LPS from E. coli). Data is presented as mean with SEM (n=6-7).

The foregoing is considered as illustrative only of the principles of the invention. Furthermore, since numerous modifications and changes will readily occur to those skilled in the art, it is not desired to limit the invention to the exact construction and operation shown and described, and accordingly, all suitable modifications and equivalents may be resorted to, falling within the scope of the invention.

### EXAMPLE 1

### Manufacturing of IL-17 Receptor Agonists

IL-17 receptor agonists can be manufactured using standard methods in molecular biology by those skilful in the art. Examples of such methods are production of IL-17 receptor agonists by human or other mammal cell lines using various types of recombinant DNA techniques, recombinant expression in other cell systems such as bacteria, yeast and viruses, though chemical synthesis or other known biotechnology methods.

### EXAMPLE 2

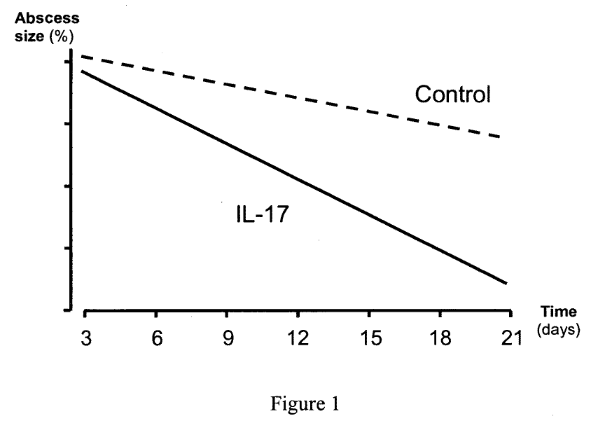

Use of IL-17 receptor agonists against local abscesses in humans in vivo Recombinant human IL-17A protein (R&D systems, Abingdon, England) is to be prepared in a sterile, pyrogen-free saline solution (10 microgram/mL in 0.9% NaCl). Forty patients with subcutaneous abscesses in their buttocks are to be recruited from an open-ward facility of general surgery at a university hospital. Twenty of these patients are to be given active treatment (IL-17A solution) and 20 patients are given vehicle (0.9% NaCl solution); the latter constituting the control subjects. The IL-17A solution is to be administered in 20 human patients at the site of the abscess, as a subcutaneous or intramuscular injection depending upon the location of the abscess, in a total dose of 3 microgram intermittently every third day until the abscess is smaller than 5 mm in diameter (ie. no longer detectable). The vehicle solution is administered every third day for 21 days. The size of the abscess is detected and measured (in cm) using ultra sonography (Model SSD500, Aloka Gmbh, Meerbuch, Germany) and its size is expressed over time as percent of size prior to treatment (%). The expected results are shown in FIG. 1.

### EXAMPLE 3

### Impact of IL-17A on the Apoptosis of Mouse Neutrophils

Blood neutrophils were obtained from mice (male Balb/c; 7-10 weeks old; Taconic, Ejby, Denmark) that were anesthetized using a mixture of ketamin (670 mg/kg, Ketalar; Pfizer, Täby, Sweden) and xylazin (130 mg/kg, Rompun; Bayer, Leverkusen, Germany) intraperitoneally. These mice were then euthanized by puncture of the left heart ventricle and blood was taken. Red blood cells were lysed and the white blood cells were washed repeatedly. Neutrophils were separated using a commercial assay (Anti-Ly-6G MicroBead Kit; Miltenyi Biotec, Bergisch Gladbach, Germany) as described in the manufacturer's manual. The number of positively selected neutrophils was counted using a Bürker-chamber. The purity of the positive selected neutrophils was determined by preparing cytospin slides that was stained in May-Grunewald-Giemsa staining; a standard method for those skilled in the art. Cell differential counts of 400 cells were performed. Using these methods, we proved the purity of neutrophils to be more than 95%.

The neutrophils were cultured at 37° C. and 5% CO₂ in supplemented medium (RPMI 1640 supplemented with 10% FBS, 1% penicillin-streptomycin, 1 mM sodium pyruvate and 2 mM L-glutamine; all products from Sigma-Aldrich) alone, or together with 1, 10, or 100 ng/mL recombinant mouse IL-17A protein (cat. No. 421-ML; R&D Systems, Minneapolis, USA). After 48 hours of incubation, the survival of the neutrophils was assessed using a commercial apoptosis assay (Annexin V-PE apoptosis detection kit; BD Biosciences, Mountain View, USA). Neutrophils were characterized using a FACScan flow cytometer (BD, Mountain View, USA) and analyzed by Cell Quest Software (BD). Results were presented as percent viable (Annexin V⁻, 7AAD⁻), apoptotic (Annexin V⁺, 7AAD⁻) and necrotic (Annexin V⁺, 7AAD⁺) neutrophils. The obtained results are shown in FIG. 2.

### EXAMPLE 4

Release of MPO by Mouse Neutrophils Stimulated with IL-17A

All procedures for neutrophil isolation and culture were identical to those presented in Example 3 above until, after 48 hours of incubation, the conditioned media were harvested and centrifuged (300 g, 10 min, +4° C.) to remove cells and debris. The cell-free supernatants were harvested and frozen (−80° C.) for later analysis of MPO, using a commercial ELISA-kit (cat. No. HK210; Hycult Biotechnologies, Uden, The Netherlands) in accordance with the product manual. The obtained results are shown in FIG. 3.

### EXAMPLE 5

Neutrophil Phagocytosis by Mouse Bronchoalveolar Macrophages Stimulated with IL-17A

Neutrophils were isolated as described in Example 3 above and were then labelled with the fluorescence marker CFDA SE (2 μM) (Vybrant® CFDA SE Cell Tracer Kit; Molecular Probes, Eugene, USA), in accordance with the product manual. These isolated and marked neutrophils were then aged by culture (1×10⁶ neutrophils/mL) in 37° C. and 5% CO₂ for 48 hours in supplemented RPMI 1640 medium.

Bronchoalveolar macrophages were harvested from anesthetized mice (see Example 3 above) by conducting bronchoalveolar lavage (BAL: 4×1 mL of Hank's Balanced Salt Solution; Sigma-Aldrich). The BAL cells were counted in a Bürker-chamber and the BAL macrophages were isolated from the rest of the BAL cells by seeding all the BAL cells (3.75×10⁵ macrophages/mL in non-supplemented RPMI 1640 medium; 200 μL/well) in 16-well chamber slides (Lab-Tek™ Chamber Slides™; Nunc, Roskilde, Denmark) at 37° C. and 5% CO₂. After this, the macrophages were let to adhere for 2 hours and non-adherent cells were then washed away. The adherent BAL macrophages were conditioned in supplemented medium (200 μL/well) containing lipopolysaccharide (LPS, E. coli, serotype 026:B6, Sigma-Aldrich) at a concentration of 1 ng/mL for 48 hours. The culture medium was then aspirated, and the macrophages were incubated with recombinant mouse IL-17A protein (1, 10, or 100 ng/mL), LPS (positive control: 1 ng/mL), or supplemented medium alone (vehicle, i.e. negative control) for 24 hours. After conditioning, the culture medium was aspirated and replaced with fresh supplemented medium, containing recombinant mouse IL-17A protein (1, 10, or 100 ng/mL), LPS (positive control: 1 ng/mL), or supplemented medium alone (vehicle, i.e. negative control), to maintain the incubation conditions. The adherent macrophages were subsequently exposed to the fluorescence-labelled and aged neutrophils (3×10⁴ cells/well). After 2 hours of incubation under these conditions, the culture medium was aspirated and neutrophils that were not phagocytosed were washed away. The remaining cells were then fixed (4% formaldehyde, 15 min, room temperature). The walls of the chamber slide were removed, and a cover glass was mounted on the slide. The nuclei of the macrophages were labelled red by adding 7AAD (BD Biosciences) to the mounting medium (prod. No. H-1400; Vector Laboratories, Burlingame, USA).

To assess the phagocytosis of neutrophils qualitatively and quantitatively, the chamber slides were photographed using a fluorescence microscope (Zeiss Axioplan 2; Carl Zeiss AG, Jena, Germany; 40× magnification), and 500 randomly selected macrophages from each well were counted. The percentage of phagocytosis was calculated by dividing the number of macrophages (red nuclei) that had phagocytosed aged neutrophils (green) by the number of counted macrophages. The obtained results are shown in FIGS. 4A and 5, respectively.

### EXAMPLE 6

### Phagocytosis of Particles in Mouse Macrophages Stimulated by IL-17A

These experiments were identical to the experiments described in Example 5 above, with the following exceptions: after conditioning and replacement of the cell medium, the mouse bronchoalveolar macrophages were subsequently exposed to fluorescent yellow-green carboxylate-modified latex beads (1 ∥m in diameter; 7.5×10⁵ beads/well; product number L 4655; Sigma-Aldrich) for 2 hours. For these experiments with fluorescence-labelled latex beads, the phagocytic index (PI) was calculated. PI was calculated as the total number of phagocytosed latex beads found in the macrophages divided by the number of counted macrophages. The obtained results are shown in FIG. 6.

### EXAMPLE 7

### Phagocytosis of Particles in Human Macrophages Stimulated by IL-17A

Human monocyte-derived macrophages were obtained from fresh blood from healthy volunteers using standard methods. Briefly, peripheral blood mononuclear cells were collected by density centrifugation over a Ficoll gradient (Ficoll-Paque Plus; Amersham Biosciences & Pharmacia Biotech, Uppsala, Sweden). The blood monocytes were then isolated by negative selection using Monocyte Isolation Kit II (Miltenyi Biotec, Bergisch Gladbach, Germany). The cell separation was performed in accordance with the product manual. To obtain a macrophage-like phenotype, the monocytes were cultured at a concentration of 1.25×10⁶ monocytes/mL (400 μL/well) in 8-well chamber slides (Lab-Tek Chamber Slides; Nunc) at 37° C. and 5% CO₂ in supplemented medium (see above) and in the presence of 10 ng/mL recombinant human GM-CSF protein (R&D Systems) for 5 days. After conditioning and replacement of the cell medium (described in detail in Example 5 above) the monocyte-derived macrophages were subsequently exposed to fluorescent yellow-green carboxylate-modified latex beads (1 μm in diameter; 4.55×10⁶ beads/well; Sigma-Aldrich) during 2 hours. The phagocytic index (PI) was then calculated. The obtained results are shown in FIG. 7.

### EXAMPLE 8

Release of MIP-2 by mouse macrophages stimulated with IL-17A All methodological procedures for the isolation and culture of mouse bronchoalveolar macrophages in vitro were identical to those presented in Example 5 above. The conditioned medium that was harvested after the last 24 hours of incubation was centrifuged (300 g, 10 min, +4° C.) to remove cells and debris. The cell-free supernatant was then frozen (−80° C.) for later analysis of MIP-2. This cytokine was analysed using a commercial ELISA-kit (cat. No. HK210; Hycult Biotechnologies, Uden, the Netherlands) in accordance with the product manual. The obtained results are shown in FIG. 8.

While the invention has been described with reference to specific embodiments, it will be appreciated that numerous variations, modifications, and embodiments are possible, and accordingly, all such variations, modifications, and embodiments are to be regarded as being within the spirit and scope of the invention.

### REFERENCES

1. Kolls J, Linden A. Interleukin-17 family members and inflammation. Immunity 2004; 21: 467-476.

2. Ivanov S, Lindén A. Th-17 cells in the lungs? Expert Review of Respiratory Medicine 2007; 1: 279-293.

3. Gaffen S L. An overview of IL-17 function and signalling. Cytokine 2008; 43: 402-407.

4. Korn T, Bettelli E, Oukka M, Kuchuroo V K. IL-17 and Th-17 cells. Annual Reviews of Immunology 2008; 27: 485-517.

5. Linden A. A role for the cytoplasmic adaptor protein Act1 in mediating IL-17 signalling. Science STKE. 2007; 398: re4.

6. Shen F, Gaffen S L. Structure-function relationships in the IL-17 receptor: Implications for signal transduction and therapy. Cytokine 2008; 41; 92-104.

7. Peric M, Koglin S, Kim S M, Morizane S, Besch R, Prinz J, Ruzick T, Gallo R L, Scauber J. IL-17A enhances vitamin D3-induced expression of cathelicidin antimicrobial peptide in human keratinocytes. Journal of Immunology 2008; 15: 181: 8504-8512.

8. Kao C Y, Chen Y, Thai P, Wachi S, Huang F, Kim C, Harper R, Wu R. IL-17 markedly up-regulates b-defensin-2 expression in human airway epithelium via JAK and NF-kB signalling pathways. Journal of Immunology 2004; 173: 3482-3491.

9. Laan M, Linden A. The IL-17 family of cytokines in Respiratory Medicine and Allergology. Recent Patents on Inflammation & Allergy Drug Discovery 2008; 2: 82-91.

10. Ivanov S, Lindén A. IL-17 as a drug target in human disease. Trends in Pharmacological Sciences 2009; 30: 95-103.

11. Pallin D J, Egan D J, Pellitier A J, Espinola J A, Hooper D C, Camargo C A. Increased US emergency department visits for skin and soft tissue infections, and changes in antibiotic choices, during the mergence of community-associated methicillin-resistant staphylococcus aureus. Annals of Emergency Medicine 2008; 51: 291-298.

12. Jahanfar S, N g C J, Teng C L. Antibiotics for mastitis in breastfeeding women. The Cochrane Database Systematic Review 2009; January 21 (1):CD005458.

13. Laan M, Cui Z H, Hoshino H, Lotvall J, Sjostrand M, Gruenert D C, Skoogh B-E, Lindén A. Neutrophil recruitment by human IL-17 via C—X—C chemokine release in the airways. Journal of Immunology 1999; 162 (4): 2347-2352.

14. Hoshino H, Laan M, Sjostrand M, Lotvall J, Skoogh B E, Lindén A. Increased elastase and myeloperoxidase activity associated with neutrophil recruitment by IL-17 in airways in vivo. Journal of Allergy and Clinical Immunology 2000; 105: 143-149.

15. Prause O, Bozinovski S, Anderson G P, Lindén A. Increased matrix metalloproteinase-9 concentration and activity after stimulation with interleukin-17 in mouse airways. Thorax 2004; 59: 313-317.

What is Claimed Is:

1. A method of treating a local infection in a mammal, comprising administering an IL-17 receptor agonist at the site of the local infection in the mammal.

2. The method of claim 1, wherein the IL-17 receptor agonist is selected from the group consisting of IL-17A, IL-17F, IL-17A/F, and other synthetic or naturally-occurring agonists at IL-17 receptors including the IL-17 A/C receptor complex and other IL-17 receptors.

3. The method of claim 1, wherein the local infection is selected from the group consisting of abscesses in skin, soft tissue and elsewhere in mammalian organs.

4. The method of claim 1, wherein the method of administration of the IL-17 receptor agonist is selected from the group of subcutaneous, intramuscular and local 1 5 administration.

5. The method of claim 1, wherein the IL-17 receptor agonist is administered to the mammal in a total amount of from 0.1-100 micrograms.

6. The method of claim 1, wherein the IL-17 receptor agonist is administered once to the mammal.

7. The method of claim 1, wherein the IL-17 receptor agonist is administered intermittently to the mammal.

8. The method of claim 1, wherein the IL-17 receptor agonist is administered repeatedly to the mammal.

9. The method of claim 1, wherein the IL-17 receptor agonist is administered continuously over time to the mammal.

10. A method of causing an increase in macrophage phagocytosis of bacteria, fungi, cellular debris and particles, comprising administering an IL-17 receptor agonist to the site of the local infection in the mammal.

11. The method of claim 10, wherein the IL-17 receptor agonist is selected from the group consisting of IL-17A, IL-17F, IL-17A/F, and other synthetic or naturally-occurring agonists at IL-17 receptors including the IL-17 A/C receptor complex and other IL-17 receptors.

12. The method of claim 10, wherein the local infection is selected from the group consisting of abscesses in skin, soft tissue and elsewhere in mammalian organs.

13. The method of claim 10, wherein the method of administration of the IL-17 receptor agonist is selected from the group of subcutaneous, intramuscular and local administration.

14. The method of claim 10, wherein the IL-17 receptor agonist is administered to the mammal in a total amount of from 0.1-100 micrograms.

15. The method of claim 10, wherein the IL-17 receptor agonist is administered once to the mammal.

16. The method of claim 10, wherein the IL-17 receptor agonist is administered intermittently to the mammal.

17. The method of claim 10, wherein the IL-17 receptor agonist is administered repeatedly to the mammal.

18. The method of claim 10, wherein the IL-17 receptor agonist is administered continuously over time to the mammal.

19. A method of causing a release of antimicrobial compounds from neutrophils, comprising administering an IL-17 receptor agonist to the site of a local infection in the mammal.

20. The method of claim 19, wherein the IL-17 receptor agonist is selected from the group consisting of IL-17A, IL-17F, IL-17A/F, and other synthetic or naturally-occurring agonists at IL-17 receptors including the IL-17 A/C receptor complex and other IL-17 receptors.

21. The method of claim 19, wherein the local infection is selected from the group consisting of abscesses in skin, soft tissue and elsewhere in mammalian organs.

22. The method of claim 19, wherein the method of administration of the IL-17 receptor agonist is selected from the group of subcutaneous, intramuscular and local administration.

23. The method of claim 19, wherein the IL-17 receptor agonist is administered to the mammal in a total amount of from 0.1-100 micrograms.

24. The method of claim 19, wherein the IL-17 receptor agonist is administered once to the mammal.

25. The method of claim 19, wherein the IL-17 receptor agonist is administered intermittently to the mammal.

26. The method of claim 19, wherein the IL-17 receptor agonist is administered repeatedly to the mammal.

27. The method of claim 19, wherein the IL-17 receptor agonist is administered continuously over time to the mammal.
